(19) United States
(12) Patent Application Publication
(10) Pub. No.: US 2013/0171273 A1
(43) Pub. Date: Jul. 4, 2013

(54) METHOD OF TREATING IMPAIRED WOUND HEALING IN DIABETICS

(71) Applicants: FAHD AL-MULLA, AL-YARMOUK (KW); MILAD BITAR, AL-YARMOUK (KW)

(72) Inventors: FAHD AL-MULLA, AL-YARMOUK (KW); MILAD BITAR, AL-YARMOUK (KW)

(73) Assignee: KUWAIT UNIVERSITY, SAFAT (KW)

(21) Appl. No.: 13/339,347

(22) Filed: Dec. 28, 2011

(51) Int. Cl. A61K 33/00; A61P 17/02; A61K 31/433

(52) U.S. Cl. 424/722; 514/363

(57) ABSTRACT

The method of treating impaired wound healing in diabetics comprises the step of administering an effective amount of a glycogen synthase kinase 3-β (GSK-3β) inhibitor to a diabetic patient in need thereof to activate the NF-E2-related factor 2 (Nrf2) and genes downstream of Nrf2 that normally regulate the expression and coordination of antioxidant responses during wound healing, but are suppressed in the diabetic patient undergoing the oxidative stress that can occur during wound healing. The GSK-3β inhibitor may be lithium or a pharmaceutically acceptable salt thereof, or TDZD-8 (4-benzyl-2-methyl-1,2,4-thiadiazolidine-3,5-dione). The method may further comprise the step of testing the diabetic patient for the presence of oxidative stress and decreased Nrf2, which enables the early or prophylactic treatment of the patient with a GSK-3β inhibitor when the patient first presents with a wound, rather than waiting for other symptoms of impaired wound healing to occur.

### BACKGROUND OF THE INVENTION

1. Field of the Invention

The present invention relates to wound healing, and particularly to a method of treating impaired wound healing in diabetics.

2. Description of the Related Art

Cutaneous wound healing involves a cycle of connective tissue matrix deposition, contraction, and epithelialization to close and heal the wound. These overlapping stages are coordinated by a cascade of cell signaling proteins that induces clotting and inflammation, followed by new proliferation and differentiation of cells. Typically, the final steps of wound healing include closure several days to two weeks after injury, which is followed by remodeling of the new tissue.

However, persons who suffer from certain chronic conditions, however, often suffer from impaired wound healing. In diabetics, for example, the process of wound healing is prolonged, or results in wounds that cannot completely close such as foot ulcers that become chronic, or only heal very slowly.

Thus, a method of assessing cutaneous wounds for treating impaired healing solving the aforementioned problems is desired.

### SUMMARY OF THE INVENTION

The method of treating impaired wound healing in diabetics comprises the step of administering an effective amount of a glycogen synthase kinase 3-β (GSK-3β) inhibitor to a diabetic patient in need thereof to activate the NF-E2-related factor 2 (Nrf2) and genes downstream of Nrf2 that normally regulate the expression and coordination of antioxidant responses during wound healing, but are suppressed in the diabetic patient undergoing the oxidative stress that can occur during wound healing. The GSK-3β inhibitor may be lithium or a pharmaceutically acceptable salt thereof, or TDZD-8 (4-benzyl-2-methyl-1,2,4-thiadiazolidine-3,5-dione). The method may further comprise the step of testing the diabetic patient for the presence of oxidative stress and decreased Nrf2, which enables the early or prophylactic treatment of the patient with a GSK-3-β inhibitor when the patient first presents with a wound, rather than waiting for other symptoms of impaired wound healing to occur. The method may further provide for testing and evaluation of the diabetic patient's oxidative stress and Nrf2 activities after the initial administration of the GSK-3β inhibitor for monitoring and adjusting the dosage of the inhibitor.

The inventors have found that the impairment of proper wound healing observed in chronic wounds, such as those suffered as a complication of diabetes, is in measure a result of impairment of Nrf2 function. This impairment is associated with an increased environment of oxidative stress. In spite of previous research demonstrating no association between Nrf2 and the rate of wound healing in healthy mammals, the inventors have surprisingly found that its deficit in the condition of diabetes impairs wound healing, and that pharmacologically activating available Nrf2 provides the unexpected benefit of improving the rate of healing of chronic cutaneous wounds and chronic ulcers. The method of treating diabetes-related impaired wound healing can include regulating upstream inhibitors of Nrf2 to achieve the desired increase in Nrf2 expression and activity.

The method can include a diagnostic method. In this embodiment, a sample of fibroblasts can be provided from the wound and the activity of Nrf2 can be assessed by a number of methods. For example, Nrf2 activates the transcription of a number of proteins and peptides that counteract excessive reactive oxygen species (ROS), which are a cause of oxidative stress. Examples of ROS-counteracting proteins are NQO1, GR, SOD2, GCLC, and GSTP1, and the mRNA level of each alone or any in combination can be used as measurement of Nrf2 activity. Nrf2 activity can also be assessed based on the fact that the kinases GSK-3β and Fyn kinase inhibit Nrf2. By measuring their activation states a practitioner can assess Nrf2 activity in a wound considered at risk of impaired healing. Activation of GSK-3β, in particular, can be measured by its phosphorylation state. Phosphorylation at Serine 9 (Ser9) in the polypeptide chain that constitutes GSK-3β causes inhibition of GSK-3β's kinase activity, whereas phosphorylation of Tyrosine 216 (Tyr216) in GSK-3β's polypeptide chain leads to activation of GSK-3β's kinase activity. Determination of upregulation of GSK-3β's kinase activity by measurement of phosphorylation at either site is indicative of decreased Nrf2 activity.

The inventors have also found that measurement of oxidative stress in conjunction with determining Nrf2 activity can aid in predicting impaired cutaneous wound healing. For example, oxidative stress in the fibroblasts provided from a wound can be measured by a cytochrome c assay, by a lucigenin assay, or by a MCB assay, which are known in the art. Furthermore oxidative stress can be determined from the levels of NOX1 mRNA and NOX4 mRNA, either separately or in combination, in the fibroblasts.

The method of the invention can also be used in treating impaired-healing cutaneous wounds. For example, the determination that the fibroblasts from a wound have levels of Nrf2 activity lower than a known standard level, and that they have an oxidative stress level that is higher than a known standard level, indicates to a practictioner of the method that he or she should treat the patient with an activator of Nrf2. The activator of Nrf2 can be an inhibitor of an upstream inhibitor. For example, the kinase activity of both Fyn kinase and GSK-3β directly or indirectly inhibit Nrf2. In particular, lithium and TDZD-8 can activate Nrf2 by inhibiting GSK-3β. A compound like lithium can be administered either systemically (i.e. orally, intravenously, intraperitoneally) or topically at the site of the wound, and can be included in a composition with pharmaceutically acceptable excipients or vehicles. These and other features of the present invention will become readily apparent upon further review of the following specification and drawings.

### BRIEF DESCRIPTION OF THE DRAWINGS

FIG. 1A is a chart showing Superoxide generation in diabetic fibroblasts (DFs), control fibroblasts (CFs), and diabetic fibroblasts that received VAS2870, a specific NADPH oxidase inhibitor.

FIG. 1B is a chart showing isolated diabetic cell membrane superoxide production and the effect of various superoxide inhibitors.

FIG. 1C is a chart showing elevation of NOX1 and NOX4 mRNA in diabetic cells.

FIG. 1D is a chart showing mitochondrial-specific reactive oxygen species in control cells and diabetic cells in the presence or absence of mitochondrial inhibitors.

FIG. 1E is a chart showing levels of lipid peroxidation in control cells and diabetic cells in the presence or absence of oxidative agents.

FIG. 1F is a chart showing the activity of plasma membrane redox system (PMRS) enzymes in control cells and diabetic cells.

FIG. 1G is a chart showing glutathione levels in control cells and diabetic cells in the presence or absence of oxidative agents.

FIG. 2A is a chart showing levels of Nrf2 mRNA in control cells in the presence of anti-sense Nrf2 treatments.

FIG. 2B is a chart showing the percent loss of viability of control cells, diabetic cells, or Nrf2 knockout cells in the presence of various concentrations of hydrogen peroxide.

FIG. 2C is a chart showing detectable caspase-3-like activity in control cells, diabetic cells, or cells treated with anti-Nrf2 oligos.

FIG. 2D is a chart showing levels of detectable ATP in control cells, diabetic cells, or cells treated with antisense Nrf2.

FIG. 2E is a chart showing the rate of release of lactate dehydrogenase (LDH) in control cells, diabetic cells, or antisense Nrf2 cells in the presence or absence of hydrogen peroxide.

FIG. 2F is a chart showing levels of inflammatory cytokines released by control cells, diabetic cells, or antisense Nrf2 cells.

FIG. 2G is a chart showing levels of mRNA transcription of inflammatory cytokines in control cells, diabetic cells, or antisense Nrf2 cells.

FIG. 3A is a chart showing total Nrf2 protein levels in control cells and diabetic cells in the presence or absence of the oxidative agent tBHQ.

FIG. 3B is a chart showing total Keap1 protein levels and the amount of Keap1 bound to Nrf2 in control cells and diabetic cells.

FIG. 3C is a chart showing Nrf2 degradation rates (protein half-life in minutes) in control cells and diabetic cells.

FIG. 3D is a chart showing Nrf2 accumulation in the nucleus of control cells and diabetic cells in the presence or absence of the oxidative agent tBHQ.

FIG. 3E is a chart showing increases in oxidative response gene mRNAs in control cells, diabetic cells, and antisense Nrf2 cells in the presence of tBHQ.

FIG. 3F is a chart showing that oligomycin treatment caused an increase in the expression of Mn-SOD, catalase, GSTP1 and GCLC but not of NQO1 in CFs, but that this phenomenon was markedly suppressed as a function of diabetes or Nrf2 knockout.

FIG. 3G is a chart showing increases in oxidative response gene transcription in control cells, diabetic cells, and antisense Nrf2 cells in the presence of oligomycin.

FIG. 4A is a chart showing the relationship between the level of expression of Fyn kinase and the levels of phospho-Ser-9 GSK-3β (inactive) and pGSK-3β-tyr-216 (active) in DFs.

FIG. 4B is a chart showing the effect of lithium on levels of nuclear Nrf2 its transcriptional activity.

FIG. 4C is a chart showing the quantity of Nrf2 purified from CFs and DFs treated with vehicle, lithium, or lithium and tBHQ binding to an ARE consensus site

FIG. 4D is a chart showing catalase mRNA levels in CFs and DFs in response to lithium alone or in combination with tBHQ or oligomycin.

FIG. 4E is a chart showing GSTP in RNA levels in CFs and DFs in response to lithium alone or in combination tBHQ or oligomycin.

Similar reference characters denote corresponding features consistently throughout the attached drawings.

### DETAILED DESCRIPTION OF THE PREFERRED EMBODIMENTS

The method of treating impaired wound healing in diabetics comprises the step of administering an effective amount of a glycogen synthase kinase 3-β (GSK-3β) inhibitor to a diabetic patient in need thereof to activate the NF-E2-related factor 2 (Nrf2) and genes downstream of Nrf2 that normally regulate the expression and coordination of antioxidant responses during wound healing, but are suppressed in the diabetic patient undergoing the oxidative stress that can occur during wound healing. The GSK-3β inhibitor may be lithium or a pharmaceutically acceptable salt thereof, or TDZD-8 (4-benzyl-2-methyl-1,2,4-thiadiazolidine-3,5-dione). The method may further comprise the step of testing the diabetic patient for the presence of oxidative stress and decreased Nrf2, which enables the early or prophylactic treatment of the patient with a GSK-3β inhibitor when the patient first presents with a wound, rather than waiting for other symptoms of impaired wound healing to occur. The method may further provide for testing and evaluation of the diabetic patient's oxidative stress and Nrf2 activities after the initial administration of the GSK-3β inhibitor for monitoring and adjusting the dosage of the inhibitor.

Impaired wound healing is often a complication of diabetes, in which a cutaneous wound fails to progress through the orderly steps of healing. Instead of the normal healing process of several days to two weeks to close a cutaneous wound, the process can take several weeks to several months; in many cases the wound enters a state of stasis in which the wound fails to close at all (a non-healing wound, the most extreme case of an impaired healing wound).

Diabetes is characterized by abnormally high levels of sugar in the blood (hyperglycemia) and a muted ability of the body to process acute infusions of sugar (impaired glucose tolerance) (see, e.g., National Institutes of Health guidelines or World Health Organization guidelines). Diabetes affects about 5-7% of the population in the United States, and about 5% of diabetics in the U.S. develop a foot ulcer each year. Chronic ulcers result from numerous physiological stresses, however. In sum, three to six million people in the United States suffer non-healing wounds each year, for various reasons.

Because of the obvious importance of wound healing in maintaining health, these signaling cascades and processes have been intensively studied, but are nonetheless incompletely understood. Among the causative factors for impaired wound healing is improperly regulated cell signaling and cytokine function at the site of the injury, resulting in improper cell behavior, including a prolonged inflammatory response and increased cell death. Oxidative stress is also believed to play an important role in the situation of diabetes. For example; high glucose causes nutritional imbalance among cells at the site of the injury and leads to increased reactive oxygen species (ROS). Reactive oxygen species production within a wound is a normal step in the process of cutaneous healing, where they function in signaling and in countering infection. However, overabundant ROS are a negative physical stress generally, and are associated with impaired wound healing. In particular, certain pathological conditions, such as diabetes, may lead to an oxidative stress environment that overwhelms the body's control of ROS.

A normal physiological response to overabundance of ROS is for the body to express anti-oxidative stress peptides and enzymes that repair chemical damage caused by excess oxidative molecules. As examples, SOD2, glutathione, and catalase are associated with a general reduction of oxidative stress. The signature enzymes and peptides of a healthy response by the body to oxidative stress are part of a protective program that is initiated, in part, by the transcription factor NF-E2-related protein 2 (Nrf2). Nrf2 is expressed throughout the body and is activated by a multitude of chemical challenges. For example, Nrf2 has been shown to protect against ethanol- or ischemic/reperfusion-induced cell death, liver necrosis following bromopropane treatment in mice, and inhalation of tobacco smoke in laboratory animals.

Nrf2 is expressed in the skin, but its role in wound healing is controversial. Nrf2 was identified as a transcription factor that is upregulated by keratinocyte growth factor (KGF), a skin-healing factor, after cutaneous injury, but genetically inhibiting Nrf2 function does not prevent otherwise healthy mice from healing at a rate indistinguishable from genetically unmodified mice.

Because of Nrf2's role in counteracting toxins, an effort has been made to identify the factors that control its activity. For example, besides KGF, mentioned above, it is also known that GSK-3β, a kinase that regulates developmental pathways, such as the Writ pathway, also influences Nrf2 transcriptional activity. Lithium is a pharmaceutical that has been known to inhibit GSK-3β's role in exacerbating bipolar disorder. Because of the knowledge that Nrf2 is inhibited by GSK-3β, and because of lithium's known role as an inhibitor of Nrf2, the potential for lithium to accelerate cutaneous wound healing was tested in normal mice. Consistent with the observations that genetically manipulating Nrf2 has no effect on the rate of wound healing, application of lithium to modify the cell signaling cascade controlling activity of normal Nrf2 also had no influence on wound healing rate in normal mice.

It has been found that a heightened state of oxidative stress associated with impairment of Nrf2 in skin fibroblasts of diabetic subjects contributes to impairment of cutaneous wound healing. These findings lend themselves to identification of cutaneous wounds at risk of impaired healing, and the treatment of patients whose wounds have been identified as possessing the relevant dysfunctions. In particular the method involves identifying a cutaneous wound at risk of impaired healing, the method comprising the steps of providing a sample of fibroblasts from a cutaneous wound of a patient; determining the activity of Nrf2 in the fibroblasts, and comparing it to a known standard level; determining the level of oxidative stress of the provided fibroblasts, and comparing it to a known standard level; wherein a determination that the activity of Nrf2 is below a known standard level, and the level of oxidative stress is above a known standard level, is indicative that the wound suffered by the patient is at risk for impaired wound healing.

The inventors have found that diabetic cutaneous tissue exhibits a decline in Nrf2, both in terms of its physical presence and its activity. The method is based on the finding that depletion of Nrf2 activity, combined with a measurable increase in oxidative stress in fibroblasts at the site of a cutaneous wound, indicates a higher risk of impaired healing of the wound. The inventors have demonstrated the notion that the lack of Nrf2 activity is correlated with the impaired wound healing by depleting Nrf2 through siRNA. Through this knockdown of expression, they have been able to recapitulate the characteristics of diabetic skin fibroblasts. Furthermore, pharmacologically enhancing Nrf2 function reestablishes expression of other proteins that ameliorate oxidative stress, and in healthy tissue, maintain a normal oxidative balance.

In healthy tissue, Nrf2 upregulates a number of antioxidative proteins in response to oxidative stress, among them NAD(P)H quinone oxidoreductase (NQO-1), glutathione-S-transferase pi (GSTP), glutathione reductase (GR), glutamyl-cysteine ligase catalytic subunit (GCLC), catalase, and manganese superoxide dismutase (SOD2 or MnSOD) as examples. In one embodiment, the cells from a cutaneous wound can be treated with tert-butylhydroquinone (tBHQ), an oxidative compound. Normal fibroblasts respond with a vigorous upregulation of transcription of anti-oxidative enzymes. As examples, NQO1 is upregulated 4-6 fold, GR is upregulated 3-4 fold, GSTP is upregulated 5-7 fold, catalase is upregulated 7-10 fold, and GCLC is upregulated 5-7 fold. In contrast, cells exhibiting an overabundance of oxidative stress and an impaired Nrf2 response show a muted upregulation of these enzymes, that is, only about a 1.5 to 2-fold increase.

The determination of lack of Nrf2 function may also be made through measurement of GSK-3β phosphorylation in fibroblasts of a chronic wound. Increasing GSK-kinase activity diminishes Nrf2's transcriptional upregulation of anti-oxidative factors. GSK-3β's kinase activity is decreased by phosphorylation of its Serine 9 (Ser9) in its polypeptide chain. Conversely, GSK-3β's kinase activity increases when it is phosphorylated at Tyrosine 216 (Tyr216) of its polypeptide chain. In diabetic fibroblasts, the phosphorylation level of Ser9 is about half that of non-diabetic fibroblasts, and the phosphorylation level of Tyr216 is about double that of non-diabetic fibroblasts. Therefore measuring phosphorylation status of either of these two residues can be indicative of Nrf2 impairment. Furthermore, higher expression of Fyn kinase (which is activated by GSK-3β) in fibroblasts from a cutaneous wound indicates that Nrf2 activity is compromised. In diabetic fibroblasts, Fyn kinase expression is about double that of non-diabetic fibroblasts.

These expression patterns are confirmed in vivo in wounds of diabetic rats. The Nrf2, GSK-3β, and Fyn kinase expression patterns observed in response to activators of oxidative stress recapitulate the patterns observed in isolated cells.

Nrf2 activity can also be determined by measuring its ability to bind to an Antioxidant Response Element (ARE) consensus site. An ARE consensus site is a DNA element that is common to the promoters of anti-oxidative stress enzymes. This site is recognized and bound by Nrf2 when Nrf2 is activated. Therefore Nrf2 activity can be determined by adding a whole cell or nucleus-only lysate prepared from fibroblasts to an assay vessel containing immobilized ARE consensus site DNA. Activated Nrf2 will bind better to the ARE consensus site. For example, the inventors have demonstrated that fibroblasts from diabetic animals, when exposed to an activator of oxidative stress like oligomycin or tert-butylhydroquinone (tBHQ), do not upregulate Nrf2 activity to the same levels as in healthy fibroblasts. This is reflected in Nrf2's ability to bind to an ARE consensus site. About 2- to 4-fold more Nrf2 from healthy cells exposed to an activator of oxidative stress binds to ARE consensus site DNA in a binding assay than does from fibroblasts isolated from a diabetic animal.

Oxidative stress in fibroblasts removed from the wound can be measured with a variety of assays known in the art. In particular, a cytochrome c assay, a lucigenin assay, or a MCB assay (which measures levels of reduced glutathione in a cell) are suitable. These assays demonstrate an increase in oxidative stress between about 40 and 80% in diabetic fibroblasts relative to control fibroblasts. Additionally, mRNA expression of NADPH oxidase 1 (NOX1) and NADPH oxidase 4 (NOX4) are indicative of oxidative stress; their levels may be increased by 2 to 5-fold in diabetic fibroblasts.

Observing the cell-signaling changes described above allows a practitioner to identify which wounds may be treated by the methods of this invention. By activating Nrf2 through blocking its upstream inhibitors, including GSK-3β and Fyn kinase, the current method allows Nrf2 in the wounds of diabetic patients to upregulate enzymes and peptides that lessen oxidative stress at the site of the wound.

The inventors have shown that activation of Nrf2 can be achieved by inhibiting its upstream regulators Fyn kinase and GSK-3β. Lithium is a well known pharmacological inhibitor of GSK-3β already used in medical practice to treat other human diseases. In addition, TDZD-8 is a known inhibitor of GSK-3β that the inventors have demonstrated is also useful in activating Nrf2 to upregulate proteins that ameliorate oxidative stress. However, any compound that is capable of inhibiting GSK-3β or Fyn kinase is suitable in the method of the invention.

Inhibitory compounds may be applied topically or given orally, intravenously, or intraperitoneally in a pharmaceutically acceptable vehicle or excipient. Lithium can be applied topically in a vehicle; the solution can be between about 0.01% and 10%, although about 8% lithium by weight to volume is a typical therapeutic concentration for a topical lithium-containing medicament. Preparations of lithium succinate and lithium gluconate in a pharmaceutically acceptable vehicle for topical application are known in the art, and in the present method are intended for use in treating human patients suffering from chronic or impaired-healing wounds. A recommended dosage of lithium would be 10-50 millimolar of a lithium salt in 30% pluronic acid applied topically directly to the wounds, as needed. A recommended dosage of TDZD-8 would be about 10-50 μM in 30% pluronic acid applied topically directly to the wound, as needed.

It is important to note that the impairment of Nrf2 can be diagnostic of potential of impaired healing in any wound, and that the method of diagnosis and treatment is not limited to those patients afflicted with diabetes.

The following examples are put forth so as to provide those of ordinary skill in the art with a complete disclosure and description of how to apply the disclosed method, and are not intended to limit the scope of what the inventors regard as their invention. The following materials and methods were followed in each of the following Examples 1 through 6, where applicable.

All animals in this study were maintained in accordance with the National Institutes of Health Guidance for the care and use of laboratory animals. Type II diabetic Goto-Kakizaki (GK) rats were produced by selective inbreeding of glucose-intolerant Wistar rats. All offspring of GK animals are similarly affected by mild hyperglycemia within the first two weeks of birth. Weight-matched male Wistar rats served as a control.

Primary rat fibroblasts were derived from dorsal skin biopsies performed on four diabetic Goto Kakizaki rats (DFs) and four age (12-14 months) and sex (female) matched Wistar control rats (CFs). After sterilization in povidine solution, the rat skin was washed in sterile water and rinsed in 70% ethanol in phosphate buffered saline (PBS). The epidermis and dermis were separated following overnight incubation in 0.25% Trypsin/EDTA at 4° C. Samples were washed, diced and digested for thirty min at 37° C. in collagenase type I (250 U/ml) dissolved in Dulbecco's modified Eagle medium (DMEM; Invitrogen) containing penicillin (100 U/ml), streptomycin (100 μg/ml), 2 mM L-glutamine and 26 mM HEPES. After collagenase treatment, the cells were dislodged, centrifuged and resuspended in medium supplemented with 10% fetal bovine serum. The cells were grown under standard conditions, and the medium was changed every three to four days. It is worthy of note that control and diabetic fibroblasts from passages 3-5 were used for the experiments and these cells were grown under normo-glycemic environment (5.5 mM glucose).

To characterize oxidative conditions of cutaneous tissue, cells in 96-well plates were washed with Krebs Ringer buffer and then incubated at 37° C. in the presence of 10 μM of dihydroethidium (DHE, Molecular Probes). Fluorescence readings were taken at Ex 530 nm and Em=595 nm over a 30 min period. The determination of GSH (reduced glutathione) was achieved using the fluorescence probe monochlorobimane (MCB, Molecular Probes). Cells were incubated with 100 μl of 40 WI MCB for 20 min at 37° C., and the fluorescence intensity was measured at Ex=390 nm and Em=460 nm. Ten μl of 0.5 mM propidium iodide (PI, Molecular Probes) was added to the well in the presence of 10 μl of 1.6 mM digitonin, and this step was used to quantify the number of cells to which the GSH level was normalized.

To study NADPH oxidase activity, cell pellets collected by trypsinization and suspended in 20 mM MOPS-KOH buffer, pH 7.4 containing 250 mM, 0.1 mM EDTA and a cocktail of protease inhibitors. The cells were disrupted by sonication on ice and the cell lysates were fractionated by centrifugation twice at 29,000×g for 15 min, discarding the supernatant each time. The pellet was resuspended in sucrose buffer and assayed for NADPH oxidase activity using lucigenin chemiluminescence or cytochrome c reduction-based assay. Briefly, membrane fractions (˜10 μg protein) were diluted in sucrose buffer/protease inhibitors and then lucigenin was added ((5 μM) in the presence or absence of 100 μM NADPH. The chemiluminescence was measured in 30 sec intervals over 5 min. The specificity of superoxide measured was confirmed by adding peg-SOD (10 units/ml). For the assessment of SOD-inhibitable cytochrome reduction, ˜100 μg membrane protein was added to a buffer containing cytochrome c (50 μM), NADPH (100 μM) and in the presence or absence of peg-SOD. After one hr, activity was measured as the SOD-inhibitable increase in absorbance at 550 nm (E_{mM}=21). Assay conditions were established confirming the linearity of superoxide production with time and protein concentrations.

Mitochondrial ROS generation was determined using MitoSox Red, a mitochondrial superoxide indicator (Invitrogen), that is selectively targeted to the mitochondria and which produces red fluorescence upon ROS oxidation. MitoSox was used according to the manufacturer's protocol and published literatures. Mitochondrial mass was assessed using the Mito Tracker Green (MTG, invitrogen), which produces a green fluorescence independent of mitochondrial membrane potential. Background fluorescence was measured from wells containing probes without cells and subtracted from respective red (Ex=530, Em=590) and green (Ex=485, Em=530) fluorescence values. Red fluorescence values were normalized to mitochondrial mass represented by averaged MTG fluorescence values from respective CFs (control fibroblasts) or DFs (diabetic fibroblasts).

To measure lipid peroxidation and plasma membrane redox system (PMRS) enzymes, a total of 10-20 μg of purified plasma membrane protein in a butler containing 50 mM Tris at pH 7.6, 0.2 mM NADH, and 0.1% Triton X-100 was analyzed in the spectrophotometric determination (340 nm) of the activities of NADH CoQ and NADH-ascorbate free radical reductase [also known cytochrome b5 reductase, following the respective additions of coenzyme Q (0.2 mM) and fresh ascorbate (0.4 mM). NQO1 activity was measured at 550 nm in solution buffer containing 5 mM. Tris-HCl at pH 7.6, 0.2 mM NADPH, 0.1% Triton X-100, 10 μM menadione and 75 μM cytochrome c with or without dicoumarol. Calculation of the activity of NQO1 was based on the difference between the uninhibited and the dicoumarol-inhibited samples. Cells were assessed for lipid peroxidation using the diphenyl-1-pyrenylphosphine (DPPP, Invitrogen) fluorescence-based assay. Briefly, cells with or without a stressor [50 μM hydrogen peroxide (HP) for 24 h at 37° C.] were incubated with 50 μM of DPPP at 37° C. in the dark for 30 min, after which they were washed twice with PBS and scraped into 150 μl of PBS. A total of 100 μl of cell solution was added to a 96-well plate. The plate was read for DPPP fluorescence (excitation at 340 nm and emission at 405 nm) in a 96-well fluorescence plate reader. The remaining 50 μl of solution was used for protein determination (BCA-based assay, Pierce).

To quantify relative cell death in control and diabetic fibroblasts, cells were seeded at 1×10⁴ cells/well on a 96-well plate. After a 24 h incubation period, the cells were exposed to various concentrations of HP in serum-free medium at 37° C. for 2 h [lactate dehydrogenase (LDH), a marker for necrotic cell death] or for 16 h (cell viability and apoptosis assay). Quantification of cell viability and caspase-3-like activity was achieved using a Cell Counting Kit-8 (Dojindo, Kumamoto, Japan) and an Apo-ONE homogenous caspase-3/7 assay (Promega), respectively. The values obtained were normalized to the vehicle-treated cells. To measure the rate of necrotic cell death, a LDH release assay was performed using a CytoTox-ONE Homogeneous Membrane Integrity Assay kit (Promega). In some experiments, cells were pre-incubated for 1 h with 2 μg of the ATPase inhibitor oligomycin before exposure to HP.

To quantify responses to oxidative stress activators, fibroblasts of control and of type 2 diabetes were treated with vehicle or the OS-inducing agents ‘t-butylhydroquinone (tBHQ, 100 μM, Sigma) or oligomycin (15 μM, Sigma) for 16 h. The Cells were processed for total RNA extraction or western blotting analysis as outlined below. Both of these compounds were dissolved in ethanol with a final concentration of 0.01%. To test for the role of GSK-3β, cells were pretreated for 8 h with lithium chloride (50 mM in DMEM) or TDZD-8 dissolved in DMSO (50 μM), whose final concentration did not exceed 0.01%.

To analyze protein expression and phosphorylation, cells were seeded in 6-well plates and treated as described above. They were washed with ice-cold PBS, scraped into PBS, and centrifuged at 1000 rpm for 5 min. The cell pellet was then subjected to subcellular fractionation. To make the total cell lysate, the cells were lysed with RIPA buffer supplemented with a protease inhibitor mixture and a phosphatase inhibitor mixture. The protein concentration was determined using BCA protein assay reagents (Pierce). Fifty micrograms of total cell lysate (or cytosolic, or nuclear) fractions were resolved on a 10% SDS-polyacrylamide gel, Western blotted, and probed with antibodies specific for Nrf2, Kcap1 or Fyn (all from Santa Cruz) or GSK-3β, GSK-3β-phosho-Ser9 (P-GSK3-β, and phosphor GSK-α/-β (pTyr279/pTyr216) (Cell Signaling). The purity of the subcellular fractions was confirmed using the anti-LDH antibody (Chemieon International) and the anti-PCNA antibody (Santa Cruz) for cytosolic and nuclear fractions, respectively. The levels of protein on a Western blot were assessed using Quantity One Image software (Bio-Rad) and normalized against suitable loading controls, including anti-β-actin antibody for total and cytosolic fractions and anti-PCNA antibody for the nuclear fraction.

Assessment of Nrf2 half-life was achieved by treating control and diabetic fibroblasts with 50 μM of cycloheximide in order to block protein synthesis. Total cell lysates were collected at different time intervals and subjected to immunoblot with an anti-Nrf2 antibody. The relative intensities of the bands were determined as described above.

To measure transcription of Nrf2 target genes and NADPH oxidase genes, RNA (1 μg) was isolated from cultured fibroblasts using Trizol reagent (Invitrogen) and reverse transcribed for 1 h at 37° C. using the High Capacity cDNA Reverse Transcription Kit. Real-time quantitative RT-PCR was performed with the TaqMan Gene Expression Assay and was normalized against 18S RNA using an ABI 7900 Real-time PCR System (Applied Biosystems). Primers and probes were designed by and purchased from Applied Biosystems. Primer efficiency and specificity were verified by amplifying standard dilutions of a probe obtained by pooling all the samples and by melting curve analysis, respectively.

To study Nrf2 binding at an ARE consensus site oligonucleotide, control and diabetic fibroblasts were stimulated with tBHQ (50 μM) and nuclear extracts were used for the determination of Nrf2 binding activity to immobilized anti-oxidant response element (ARE) using a TransAM Nrf2 kit (Active Motif). Briefly, nuclear extract protein (−5 μg) was incubated in a 96-well plate containing immobilized consensus Nrf2 binding site. Wells were washed three times, and bound Nrf2 was detected by Nrf2 antibody and secondary antibody conjugated with horseradish peroxide. The signal was detected spectrophotometrically at 450 nm.

To compare the specific effects of loss of Nrf2 to characteristics observed in diabetic fibroblasts, expression of Nrf2 was inhibited in matched Wistar rat dermal fibroblasts by small-interfering RNA (siRNA) oligonucleotides (“Nrf2 knockout fibroblasts”). The siRNA sequences were designed and synthesized by Qiagen. The best silencing efficiency was obtained by incubating 2.0×10⁵ cells/well in a 6-well plate with complexes formed by 5 nM siRNA (1 μl) and 9 μl of HiPerfect transfection reagent (Qiagen) dissolved in 90 μl medium, according to the manufacturer's instructions. The transfection was achieved by adding 0.9 ml of medium to the seeded cells followed by 100 μl of siRNA/HiPerfect complex. Twenty-four hours later, 1 ml of fresh medium was added; 48 h after transfection the cells were exposed to either vehicle or the OS-inducing agents, including HP, oligomycin or tBHQ. Knock-out efficiency was verified by real-time PCR and Western blot.

To study effects on inflammatory cytokine expression, control and diabetic fibroblasts were seeded on a 6-well plate at 2.5×10⁵ per well. After incubation overnight, the cells were treated with or without 50 μM HP in serum/phenol free medium. After incubation for 16 h, supernatants were collected and analyzed for key inflammatory cytokines including tumor necrosis factor α (TNF-α), IL-1β, fractalkine (FKN) and monocyte chemoattractant protein-1 (MCP1) using commercially available ELISA kits specific for rats and according to the protocols provided by the manufacturers (R&D and Ray Biotech).

All data were expressed as means±SEM. Comparison between two groups were conducted using Student t tests. ANOVA was used to compare differences among multiple groups, followed by Tukey post hoc test for significance. A probability value of P≦0.05 was considered statistically significant. All experiments were performed in triplicate on at least three separate occasions.

### Example 1

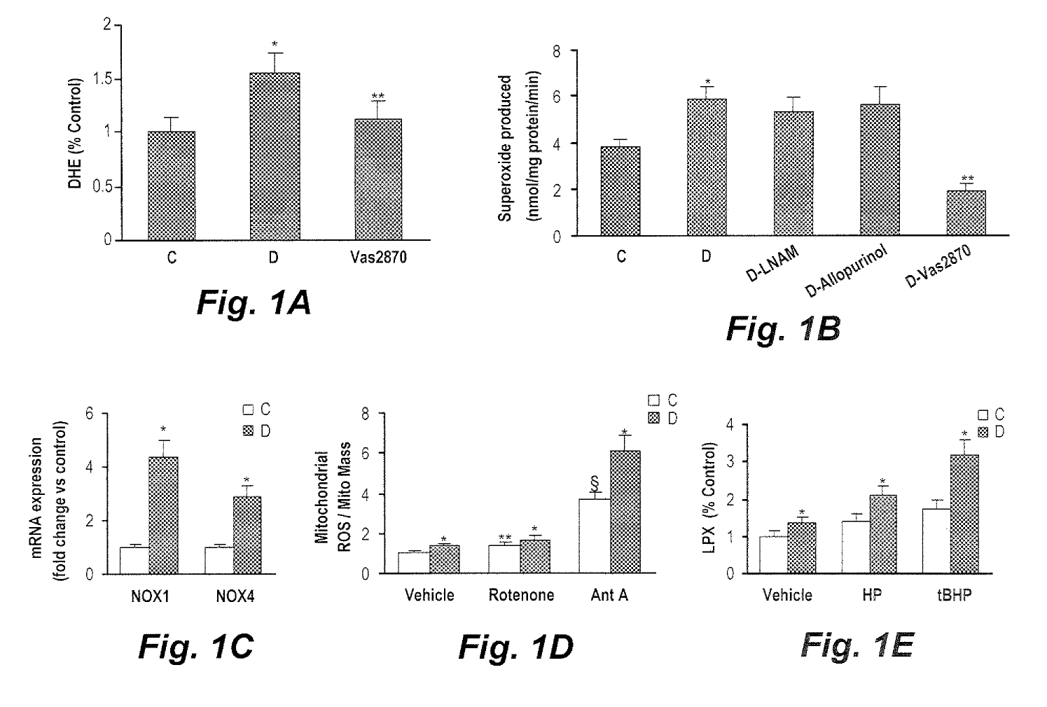

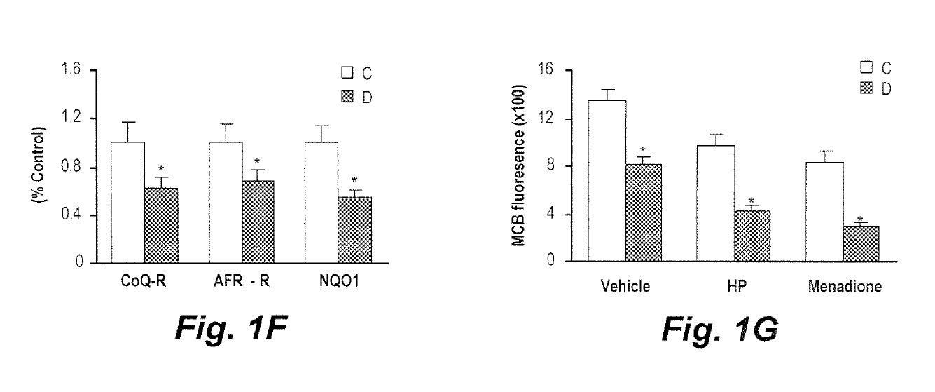

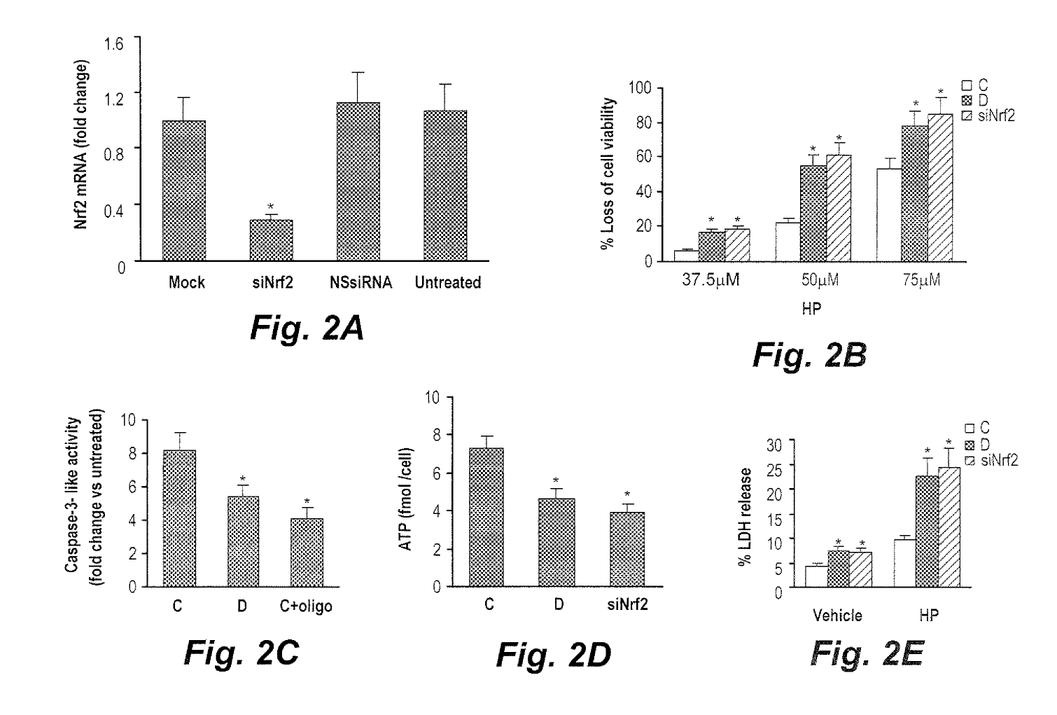

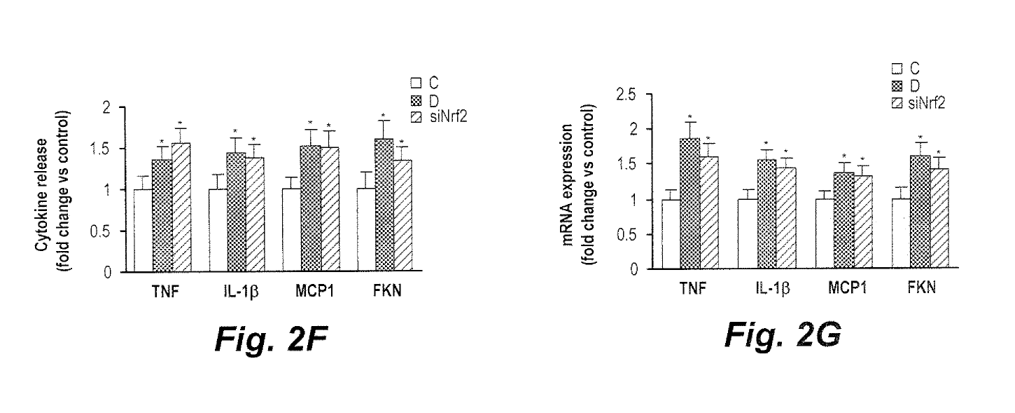

Example 1 is a study demonstrating that diabetic fibroblasts exhibit a state of heightened oxidative stress and that NADPH oxidase in the mitochondrial membrane contributes to this state. Following the procedures above, it was determined that superoxide generation over a 30-minute period was 55% higher in DFs relative to corresponding control values (FIG. 1A). This radical is a by-product of mitochondrial respiration and enzymatic oxidases. Accordingly, it was examined whether the observed elevation in superoxide stemmed from enhanced activity of the non-phagocytic NAD(P)H oxidase. The resulting data showed that VAS2870, a specific NADPH oxidase inhibitor, reduced the diabetes-related increase in superoxide by about 31%, thus supporting the partial involvement of NAD(P)H oxidase (FIG. 1A). To more directly assess the involvement of NADPH oxidase in diabetes-related increase in superoxide generation, we determined NADPH-dependent superoxide generation in 28,000×g membrane fractions of control and diabetic fibroblasts using lucigenin chemiluminescence, or the SOD-inhibitable cytochrome c reduction-based assay, DFs produced superoxide at a rate of 6.36 nmol/mg protein/min, which was significantly higher than corresponding control values (FIG. 1B). Adding various inhibitors of nitric oxide synthetase (e.g., L-NAME) or xanthine oxidase (e.g., allopurinol) did not have a significant effect on superoxide production (FIG. 2B). However, the specific oxidase inhibitor VAS2870 reduced activity by about 87%, thus confirming that NADPH oxidase activity is indeed upregulated during diabetes (FIG. 1B). Consistent with these data, we also documented that the levels of expression of mRNAs encoding for NADPH oxidase 1 (NOX1) and NADPH oxidase 4 (NOX4) were markedly augmented in DFs when compared to corresponding control values, by about 4-fold for NOX1, and about 3-fold for NOX4 (FIG. 1C).

To determine if mitochondria also contributed to the elevated ROS levels seen in DFs, the mitochondria-targeted superoxide-sensitive fluorophore MitoSOX Red (Molecular Probes) was used. For these studies, parallel measurements using 0.1 μM Mito-Tracker Green (MTG, Molecular Probes), a probe that selectively stains the mitochondria, were performed to assess total mitochondrial mass. The data derived from these studies demonstrate that mitochondrial superoxide generation when normalized to MTG fluorescence is higher in DFs relative to corresponding control values (FIG. 1D). This phenomenon was also observed in the presence of mitochondrial electron transport chain (mETC) inhibitors rotenone (complex I) and antimycin A (complex III) (FIG. 1D). However, the relative difference in ROS production between CFs and DFs is potentiated to a greater extent by antimycin A than by rotenone. Consistent with these data, it was also found that protein carbonyl levels, a measure of ROS-mediated protein oxidation, in the mitochondrial fraction are also elevated as a function of diabetes (nmol/ing protein, CFs, 1.32±0.15, DFs, 1.71±0.16, P≦0.05). Overall, the above data indicate that an upregulation in NADPH oxidase activity in connection with a defect in mETC contribute to the elevation in ROS levels during diabetes.

To monitor whether the diabetes-induced elevation in intracellular levels of ROS may reflect an increase in lipid peroxidation, a marker of accumulative oxidative stress (OS), the lipid peroxide formation of CFs and DFs at baseline and under stressed conditions was measured using the dye DPPP, which intercalates and reacts with lipid hydroperoxides. As shown in FIG. 1E, there was about a 37% enhancement in DPPP fluorescence in unstressed DFs when compared to corresponding controls. HP and t-BHP each elicited an enhancement in lipid peroxidation, which was markedly higher in diabetic than in control cells.

In view of the above data documenting an enhancement in the rate of lipid peroxidation in diabetic fibroblasts, the activity levels of key enzymes in PMRS, including coenzyme Q (CoQ) reductase, cytochrome b5 reductase and NQO1, were examined. The PMRS appears to protect against plasma membrane lipid peroxidation triggered by exogenous and endogenous OS. Consistent with the observed abnormalities in the plasma membrane lipid peroxidation during diabetes, it was also found that in this disease state the levels of PMRS-based enzymes, including CoQ-R, cytochrome b5-R and NQO1, are reduced by about 37%, 31% and 45%, respectively (FIG. 1F).

The reduced form of glutathione (GSH) represents a key component of the antioxidant defense mechanism, and this system was assessed in the cultured fibroblasts using the fluorescence probe MCB. GSH levels in DFs are reduced by about 40% compared to control counterparts (FIG. 1G). Moreover, it was also found that the sensitivity of this ROS scavenging system to various forms of OS, including HP and menadione is markedly enhanced in DFs (FIG. 1G).

To this end, the aforementioned data are consistent with the notion that an imbalance between oxidant-producing systems and antioxidant defense mechanisms appears to exist during diabetes. This phenomenon may trigger cell damage by oxidizing (as has been shown above) macromolecular structures (lipids, proteins and DNA) and modifying their functions, leading ultimately to cell death.

### Example 2

Example 2 is a study of the effect of hydrogen peroxide (HP)-induced oxidative stress on cell death among control and diabetic fibroblasts. A wealth of evidence indicates that chronic oxidative stress, which the above examples clearly confirm exist in DFs, can alter the sensitivity and the mechanism by which a cell dies in response to various stressors. Accordingly, in this study, the effect of HP, the most common endogenous oxidant, on cell viability and caspase-3-like activities in CFs and DFs was evaluated.

The resulting data show that exposure of DFs to 37.5, 50 and 75 μM HP for 16 h leads to a 17%, 55% and 78% loss in cell viability, respectively (FIG. 2A). However, exposure of control cells to the same concentrations of HP results in less marked changes in cellular viability of only 6%, 22% and 53%, respectively (FIG. 2A).

Next, the caspase-3-like activity in response to 50 μM of HP was also determined. As shown in FIG. 2B, the fold increase in caspase-3-like activity in DFs (5.4-fold) is less than that of corresponding controls (8.2-fold). In view of these data and the well-known concept that cell death by apoptosis involves a number of energy-dependent steps including the activation of caspase-3 enzyme, the intracellular level of ATP was assessed and found to be decreased as a function of diabetes (FIG. 2C). Interestingly, pre-treatment of CFs with a 2-μg dose of oligomycin, an inhibitor of ATP synthesis, like that of diabetes, suppresses the ability of HP to enhance caspase-3-like activities (FIG. 2C). While not wishing to be bound by theory, this diabetes-related decrease in intracellular free ATP level may stem from an abnormality in the mitochondrial function, as evidenced by the increase in mitochondrial ROS generation (FIG. 1D), elevated mitochondrial protein carbonyl levels, and the decrease in the activity of complex 1 of the mETC (nmol/mgprotein/min, CFs=24±3.9, DFs=13.9±2.2, P≦0.05). To this end, the above data advance the notion that CFs are more resistant than DFs to HP-induced cell death. Moreover, the enhanced cell death seen in DFs appears to be associated with a marked reduction in ATP level. Credence for this proposition is reflected by the data depicted in FIG. 2D, showing that in response to HP, the rate of LDH release into cell culture media, a measure of necrotic cell death, is markedly enhanced in DFs when compared to corresponding control values. This finding was confirmed using the propidium iodide intake-FACS-based technique. Further experimentations confirmed that this diabetes-related increase in cell death is accompanied by a marked increase in the expression and rate of release of pro-inflammatory cytokines including TNF-α, IL-1β, fractalkine and MCP1 (FIGS. 2F and G).

### Example 3

Example 3 is a study undertaken to explore the cell-signaling basis for the phenotype observed above in DFs. To inspect at the molecular level the reasons for the heightened level of oxidative stress (OS) and the enhanced sensitivity of DFs to HP-induced cell death, focused was placed on the Nrf2 signaling pathway.

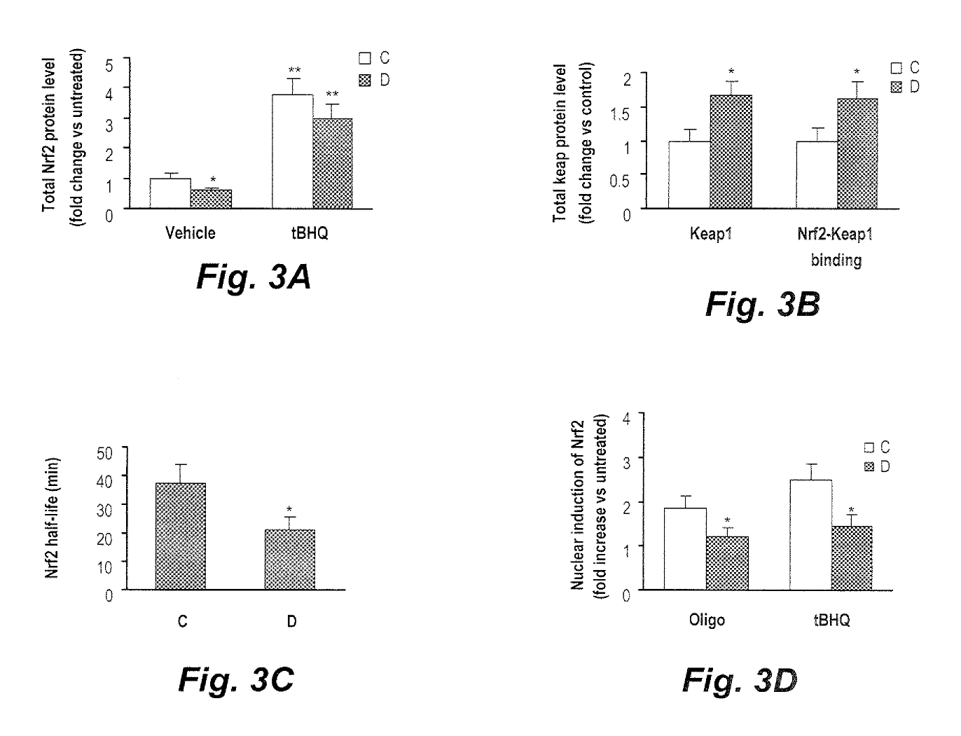

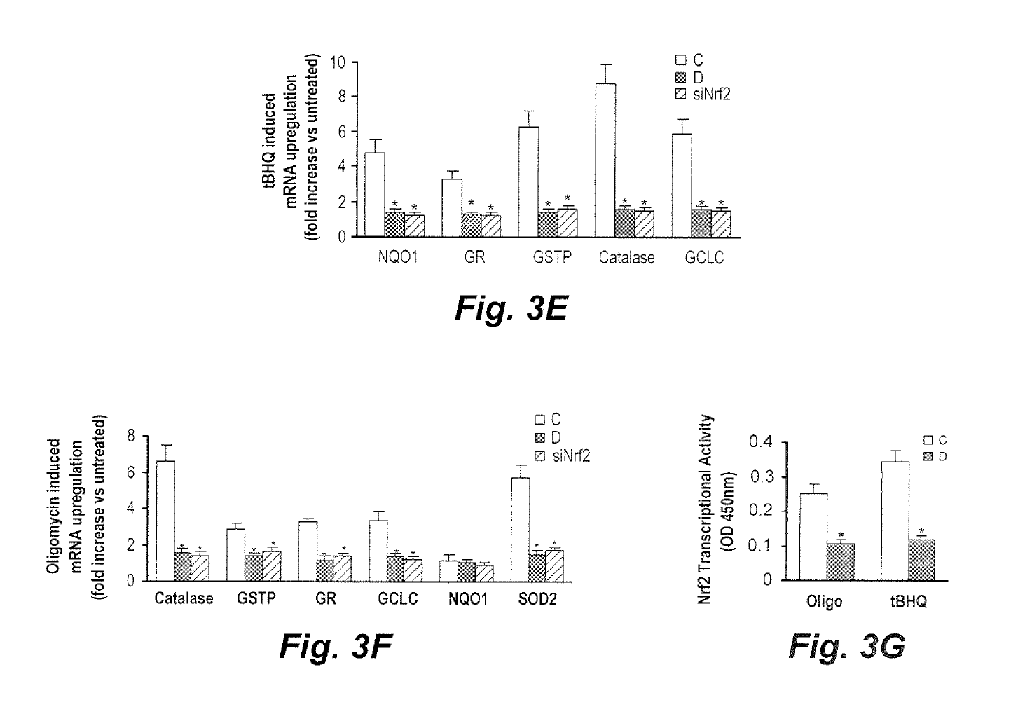

The data derived from these studies show that Nrf2 levels in total cellular protein extracts are diminished in DFs relative to CFs (FIG. 3A), an abnormality that appears not be due to a reduction in mRNA level (normalized to 18S RNA and expressed as a fold change vs. control, CFs=1±10.12, DFs=1.71±0.21, P≦0.05), nor to a decrease in mRNA half-life as shown by an experiment using actinomycin D (ActD 10 ng/ml, data not shown). These findings, in connection with the confirmed elevation in Keap1 levels (FIG. 3B), which is a component of an E3 ubiquitin ligase complex that targets Nrf2 for degradation during diabetes, prompted the assessment of Nrf2 protein stability using the CHX chase-based analysis. In these experiments, Nrf2 was allowed to accumulate to a well-detectable level in CFs/DFs using MG-132, an inhibitor of the process of 26S proteasome-mediated Nrf2 degradation. One hour later, and after extensive washing, the de novo synthesis of Nrf2 was blocked with CHX for the indicated time periods. The data presented in FIG. 3C clearly show that in DFs, Nrf2 was degraded by the proteasome at a much higher rate than that of CFs. Moreover, it was also found in a co-immunoprecipitation analysis that the degree of association of Nrf2 with Keap1 is markedly increased as a function of diabetes (FIG. 3B). Overall, the findings are consistent with the concept that, at least in fibroblasts; diabetes reduces Nrf2 protein stability, possibly by augmenting ROS-Keap1-dependent signaling pathway.

Next, the nuclear localization of Nrf2 in response to OS induced by oligomycin and tBHQ was investigated. Oligomycin-related inhibition of ATPase appeared to produce an over-reduction of the mitochondrial quinone pool, with a concomitant increase in superoxide (SO) generation. Similarly, tBHQ may undergo redox cycling, either by cellular quinone reductases, or through auto-oxidation reactions resulting in the formation of HP. The data confirmed that in the nuclei-enriched fraction of CFs, Nrf2 is markedly elevated in response to oligomycin and tBHQ (FIG. 3D). A far smaller increase in the nuclear accumulation of Nrf2 is evident in DFs (FIG. 3D). Interestingly, tBHQ elevates the level of Nrf2 in total cell lysates of control and diabetic fibroblasts to about the same extent (FIG. 3A).

To assess whether the impairment in OS-mediated nuclear translocation of Nrf2 in DFs might affect ARE-responsive genes, mRNA levels of key genes that are regulated by Nrf2 were measured using real-time PCR. In CFs, the levels of expression of catalase, GSTP1, GCLC, and NQO1 in response to tBHQ were increased by 8.8-fold, 6.3-fold, 5.7-fold, and 4.8-fold, respectively (FIG. 3E). However, in DFs, enhancement of the aforementioned Nrf2-dependent transcripts is greatly reduced. Indeed, the percent increase was in the range of 32%-78% (FIG. 3E). There were no observable changes in the levels of either Cu/Zn SOD or Mn-SOD (SOD2). Oligomycin treatment also caused an increase in the expression of Mn-SOD, catalase, GSTP1 and GCLC but not of NQO1 in CFs (FIG. 3F). This phenomenon is markedly suppressed as a function of diabetes (FIG. 3F). These results corroborate very well with the data depicted in FIG. 3G showing that the oligomycin- or tBHQ-induced increase in the transcriptional activity of Nrf2, assessed using an immobilized oligonucleotide containing the ARE consensus binding site, is markedly suppressed as a function of diabetes (FIG. 30).

Overall, the results suggest that a defect in Nrf2 signaling pathway may contribute to the increased sensitivity of DFs to OS-induced necrotic inflammation and cell death, and additionally, knockout of Nrf2, in control fibroblasts recapitulates the diabetic cell phenotype.

### Example 4

Example 4 demonstrates that knockout of Nrf2 in control fibroblasts recapitulates the diabetic cell phenotype. To confirm the participation of Nrf2 in the increased sensitivity of DFs to HP-induced necrotic cell death, RNA silencing experiments were performed. CFs were transfected with a silencing RNA (siRNA) sequence directed against Nrf2 mRNA, and the effectiveness of this strategy was evaluated using a real-time PCR-based technique.

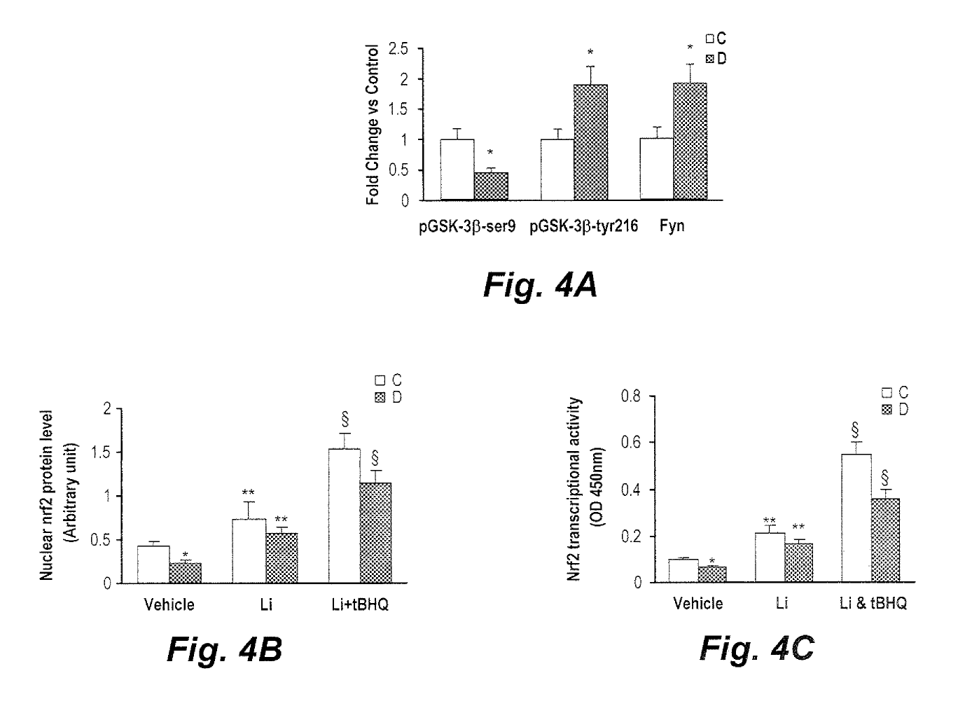

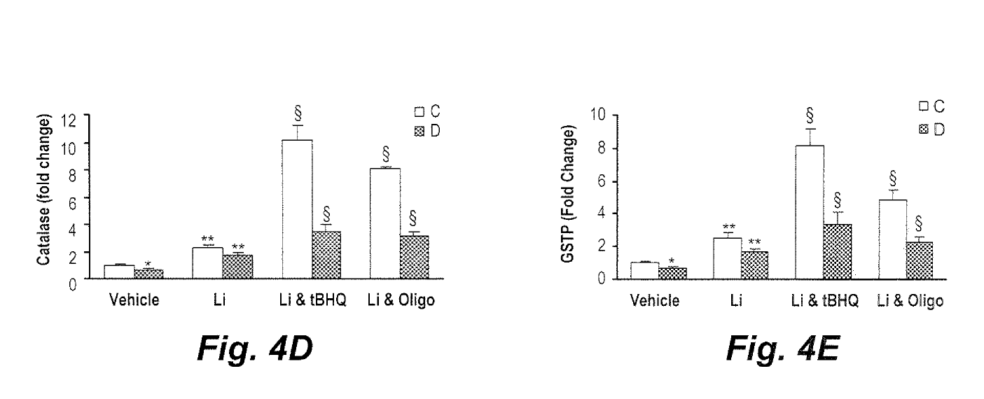

The data show a significant reduction in Nrf2 mRNA at 24 h following transfection, and this effect continues for up to 48 h (FIG. 4A). A parallel experiment was conducted with a commercially available siRNA directed against GAPDH to control for transfection and silencing efficiency. A marked decrease in the rate of GAPDH mRNA expression is evident at 24 and 48 h post-infection (data not shown). As a control for the silencing specificity and for off-target effects, cells were also transfected with a commercially available non-silencing siRNA-like sequence (NS-siRNA), which does not recognize any eukaryotic sequence (FIG. 4A). Following this confirmation step, the Nrf2 knockout fibroblasts and their control counterparts were exposed to various concentrations of HP. The data reveal that the degree of loss in cell viability, as well as the rate of release of LDH at each of the HP concentrations, is significantly higher in Nrf2 knockout fibroblasts relative to corresponding controls (FIGS. 2A and D). This phenomenon is associated with a greater accumulation of several inflammatory cytokines following HP treatment (FIGS. 2F and G). Further experiments also showed that Nrf2-knockout fibroblasts, much like DFs, exhibit a significant decrease in the various Nrf2-related genes, both at the basal level and in response to tBHQ or oligomycin (FIGS. 3E and F).

### Example 5

Example 5 is a study exploring regulation of Nrf2 by upstream kinases, and its role in fibroblast response to OS. The notion was explored that GSK-3β inhibits the nuclear accumulation of Nrf2 in response to OS via a Fyn-dependent mechanism. This sequence of events was examined in DFs because in these cells the nuclear accumulation of Nrf2 was markedly diminished. Since the activity of GSK-3β is regulated negatively by the phosphorylation of serine 9 (pGSK-3β-ser-9), and positively by the phosphorylation of tyrosine 216 (pGSK-3β-tyr-216), the expression/phosphorylation status of GSK-3β between the control and diabetic fibroblasts was compared. As shown in FIG. 4A, the degree of GSK-3β inactivation, as determined by the level of phospho-Ser-9 GSK-3β, is markedly reduced in DFs when compared to corresponding control values. In contrast, an increase in the level of pGSK-3β-tyr-216 is evident in these cells (FIG. 4A). This diabetes-related enhancement in the GSK-3β activity is associated with a significant increase in the level of expression of Fyn kinase (FIG. 4A), a downstream target of GSK-3β and an important enzyme in the control of nuclear export and degradation of Nrf2.

It was also explored whether pharmacological and siRNA-mediated down-regulation of GSK-3β activity could ameliorate the defect in Nrf2-dependent signaling during diabetes. In particular, it was analyzed whether GSK-3β inhibitors have any effect on the nuclear accumulation of Nrf2 in DFs. The most established inhibitors of GSK-3β, including lithium (IC₅₀ 2 mM) and thiadiazolidinone TDZD-8 (IC₅₀ 2 μM), were used, with the resulting data confirming that both the basal and inducible levels of nuclear Nrf2 are partially normalized in response to the aforementioned treatment (FIG. 4B, only shown for lithium). Consistent with these data, it was also confirmed that the pharmacological inhibition of GSK-3β partially restores the transcriptional activity of Nrf2, as well as the sensitivity of Nrf2-dependent genes to OS-inducing agents, including tBHQ and oligomycin (FIG. 4C-E).

In order to obtain additional evidence for the importance of GSK-3β in diabetes-induced impairment in the Nrf2 signaling pathway, and to avoid potential artifacts induced by inhibitory drugs, the level of GSK-3β was downregulated using siRNA. Silencing of GSK-3 in DFs recapitulates most of the changes seen with lithium treatment, including the increase in Nrf2 nuclear accumulation, Nrf2 transcriptional activity, and the expression of Nrf2-dependent genes.

### Example 6

This study confirmed the cell signaling, gene transcription, and protein expression patterns observed in isolated fibroblasts in in vivo wounds. To determine whether the defect in the Nrf2 signaling pathway in DFs is a phenomenon seen only in cell culture, we assessed the basal and tBHQ-induced nuclear accumulation of Nrf2 in in vivo 7-day control and diabetic wounds. It was confirmed that there is a significant decrease in the basal and OS-mediated accumulation of Nrf2 in the nuclei of 7-day diabetic wounds, as compared to control wounds. In addition, patterns of transcription of downstream targets of Nrf2 mirrored those observed in vitro. Consistent with these data, it was also found that the level of expression of Fyn and the activity of GSK-3β, as reflected by the reduced level of P-GSK-33, are markedly elevated in the diabetic wounds.

Overall, the data support the unexpected finding that an augmentation in GSK-3β/Fyn signaling reduces the nuclear accumulation and activity of Nrf2 and its downstream transcriptional targets in the diabetic state, as well as in cultured normal and diabetic dermal fibroblasts under both basal conditions (including endogenous OS) and in response to artificially induced OS.

It is to be understood that the present invention is not limited to the embodiment described above, but encompasses any and all embodiments within the scope of the following claims.

1. A method of treating impaired wound healing in diabetics, comprising the step of administering an effective amount of a glycogen synthase kinase 3β(GSK-3β) inhibitor to activate NF-E2-related factor 2 (Nrf2) and genes downstream from Nrf2 for regulating antioxidant response to wound healing in a diabetic patient in need thereof, wherein said GSK-3β inhibitor is lithium.

2-15. (canceled)

16. The method of treating impaired wound healing according to claim 1, wherein said administering step comprises administering the GSK-3β inhibitor to the patient orally.

17-18. (canceled)
